(19) United States
(12) Patent Application Publication
(10) Pub. No.: US 2015/0038758 A1
(43) Pub. Date: Feb. 5, 2015

(54) PREPARATION METHOD OF PLATINUM/TIN/METAL/ALUMINA CATALYST FOR DIRECT DEHYDROGENATION OF n-BUTANE AND METHOD FOR PRODUCING C4 OLEFINS USING SAID CATALYST

(71) Applicant: SAMSUNG TOTAL PETROCHEMICALS CO., LTD., Seosan-si (KR)

(72) Inventors: Gle PARK, Yongin-si (KR); Yeon Shick YOO, Seosan-si (KR); Jin Suk LEE, Seoul (KR); Ho Sik CHANG, Daejeon (KR); Chang Hyun CHOI, Seoul (KR); In Kyu SONG, Seoul (KR); Hyun SEO, Daejeon (KR); Jong Kwon LEE, Daejeon (KR)

(21) Appl. No.: 14/341,917

(22) Filed: Jul. 28, 2014

(30) Foreign Application Priority Data
Jul. 30, 2013 (KR) 10-2013-0090456

(51) Int. Cl. B01J 23/62; B01J 23/63; C07C 5/32

(52) U.S. Cl. CPC B01J 23/626; C07C 5/325; B01J 23/63; C07C 2523/56
USPC 585/660; 502/334; 502/329; 502/303; 502/304; 502/330

(57) ABSTRACT

The provided is a method for preparing a platinum-tin-metal-alumina catalyst by comprising: as an active ingredient, platinum which has a high activity in a direct dehydrogenation reaction of n-butane, tin which can increase the catalyst stability by preventing carbon deposition; additionally metal for reducing the level of catalyst inactivation over the reaction time; and an alumina carrier for supporting said components. Further, provided is a method for producing a high value product, C4 olefins from low cost n-butane by using the catalyst prepared by the method according to the present invention in a direct dehydrogenation reaction.

### CROSS-REFERENCE TO RELATED APPLICATION

This application claims the benefit and priority of Korean Patent Application No. 10-2013-0090456 filed Jul. 30, 2013. The entire disclosure of the above application is incorporated herein by reference.

### TECHNICAL FIELD

The present invention relates to a method for preparing a catalyst for direct dehydrogenation of N-butane, specifically to a method for preparing a platinum-tin-metal-alumina catalyst by a sequential impregnation method of various metals, tin and platinum with the use of an alumina carrier, and a method for producing C4 olefins from n-butane using said catalyst.

### BACKGROUND OF THE INVENTION

In the petrochemical industry, the light olefin manufacturing industry such as ethylene, propylene and butadiene is one of the national key industries. In this regard, production and securing of light olefins which are the basic raw materials for producing polyethylene (PE), polypropylene (PP), styrene butadiene rubber (SBR), butadiene rubber (BR), acrylonitrile butadiene styrene (ABS), styrene butadiene rubber latex (SBL) and the like, i.e. polymer products in globally increasing demand are urgently needed. Among them, the raw materials for PE and PP preparation are relatively easily secured; however, regarding the fact that n-butene and 1,3-butadiene which are also basic raw materials do not have certain supply sources and an extension of ethane cracker equipment in middle east and US recently made, there is a concern for a long-term imbalance between supply and demand of C4 olefins these days.

Among C4 olefins, currently more than 90% of butadiene is extracted from C4 fractions, which contains 44% butadiene on the average. In the past from 1940s to 1970s, a butadiene production method by dehydrogenation of butene and on-purpose butadiene production method comprising two steps for converting butane→butene→butadiene, was generally used, however these methods became uneconomical upon the energy cost increase. Now, C4 olefin production including butene, butadiene or the like is made by a naphtha cracking center (NCC) which is operated mostly under high temperature reaction conditions at more than 800° C. C4 light olefins from a naphtha steam cracking process are separated in the order of 1,3-butadiene, isobutylene, 1-butene, etc. depending on its value and benefits in a separation process, from C4 fractions which are obtained after separating C2, C3, C5+ materials from naphtha cracker. However, since the main purpose of a naphtha cracking process is to produce basic fractions such as ethylene, propylene or the like, and is not a single process for the production of n-butene and 1,3-butadiene, it is not suitable for coping with the rapidly increasing demand for n-butene and 1,3-butadiene. Further, due to the price increase of naphtha which is a current resource for obtaining C4 fractions, an extension has been mainly made for an ethane cracker, rather than a naphtha cracker, making the expansion of C4 light olefin production far more limitative. Generally, C4 fraction production yield is in the order of 9% in a naphtha cracker and 3% or so in an ethane cracker. Therefore, a method for producing C4 light olefins from butane, not from the existing naphtha cracking equipment has been required, and one of the methods, a dehydrogenation reaction in which C4 olefins are obtained by detaching hydrogen from n-butane has been gaining attention as a single process for C4 olefin production being capable of quickly coping with the recent market change and related studies have been being made (Non-patent documents 1-6).

The dehydrogenation reaction of n-butane in which hydrogen is removed from n-butane, thereby producing n-butene and 1,3-butadiene may be classified to two methods: a direct dehydrogenation reaction in which hydrogen is directly removed from n-butane; and an oxidative dehydrogenation reaction in which hydrogen is removed from n-butane through the use of oxygen, wherein the oxidative dehydrogenation reaction of n-butane is an exothermal reaction and produces stable products after the reaction, thereby being advantageous in terms of thermodynamics, however it further produces side products such as carbon monoxide and carbon dioxide, etc. through the oxidation reaction using oxygen and is disadvantageous in terms C4 olefin selectivity and yield, as compared to the direct dehydrogenation of n-butane. In the meanwhile, the direct dehydrogenation of n-butane requires as an endothermic reaction, higher temperature reaction conditions as compared to the oxidative dehydrogenation reaction, and the use of a precious metal catalyst such as a platinum catalyst, which has short catalyst life in many cases and thus needs further regeneration process. However, although said problems, this is known as a beneficial process in terms of C4 olefin selectivity and yield (Patent document 1-4, Non-patent document 7-11).

Therefore, if a direct dehydrogenation process, rather than a naphtha cracking process, is commercialized, it will be possible to produce C4 olefins through a single process, and obtain an energy-saving effect. In the meanwhile, as mentioned above, although the direct dehydrogenation of n-butane is advantageous in C4 olefin selectivity and yield, as compared to oxidative dehydrogenation reaction, problems such as short catalyst life, and occurrence of inactivation owing to coking deposition in the progress of the reaction are expected. In this regard, in order to produce C4 olefins with a high yield, a catalyst process with a high efficiency and long-term use should be studied in advance, which can provide high selectivity and prevent the catalyst from being inactivated caused by coking deposition while maintaining a high conversion rate of n-butane.

Up to now, as for the catalyst systems used for producing C4 olefins by a direct dehydrogenation of n-butane, there have been platinum-alumina based catalyst (Patent documents 1-4 and Non-patent documents 7-10), chrome-alumina based catalyst (Patent documents 5-6 and Non-patent document 7), vanadium based catalyst (Non-patent documents 12-13), etc. From late 1930s, a dehydrogenation reaction of a paraffin material for olefin production has been studied. The dehydrogenation reaction of n-butane started to be studied with the development C4 olefin production process from n-butane by using a chrome-alumina based catalyst for increasing an octane number in octane production during the Second World War. From 1960s, a dehydrogenation process of n-butane using a platinum-alumina catalyst which is based on a precious metal, platinum, has been consistently developed and researched, and from the 2000s, vanadium based catalyst have been researched, with a purpose for substituting the expensive precious metal. Among the above-described catalysts of prior arts, a platinum-alumina based catalyst are known to have the highest activity and be suitable in a direct dehydrogenation reaction of n-butane among platinum-alumina based catalyst (Non-patent document 7).

The above-described platinum-alumina catalyst is generally prepared in the form wherein platinum is supported by alumina. Specifically, a direct dehydrogenation reaction of n-butane using 0.2 g platinum-alumina catalyst prepared by using a conventional alumina carrier (γ-Al₂O₃) for supporting platinum, performed under the conditions of a feed ratio of hydrogen:n-butane=1.25:1, the total flow rate of 18 ml·min⁻¹ and a reaction temperature of 530° C. was reported, and results obtained after 10 minutes of reaction were n-butane conversion rate of 45%, C4 olefin selectivity of 53%, and yield of 24%, and after 2 hours of reaction were n-butane conversion rate of 10%, C4 olefin selectivity of 50%, and yield of 5% (Non-patent document 14).

Generally, an enhancer is often used in a platinum-alumina catalyst, and in this case, the activity may be improved by changing various conditions, depending on the interactions among platinum, an enhancer, and an alumina carrier. Particularly, as for an enhancer for platinum activity and stabilizer, tin is majorly used, and a platinum-tin-alumina catalyst obtained by supporting platinum and tin to an alumina carrier is reported to exhibit good activity in direct dehydrogenation of n-butane. Specifically, for example, it was reported that a direct dehydrogenation reaction of n-butane by using 0.2 g of a platinum-tin-alumina catalyst prepared by sequentially supporting platinum and tin to a conventional alumina carrier (γ-Al₂O₃) was performed under the conditions: a feed ratio of hydrogen:n-butane=1.25:1; the total flow rate of 18 ml·min⁻¹; and a reaction temperature of 530° C., and results obtained after 10 minutes of reaction were n-butane conversion rate of 43%, C4 olefin selectivity of 78%, and yield of 34%, and after 2 hours of reaction were n-butane conversion rate of 13%, C4 olefin selectivity of 86%, and yield of 11% (Non-patent document 14). Further, a document using copper and palladium, not tin, in a platinum-alumina catalyst as an enhancer (Non-patent document 15) was reported, wherein 0.1 g of each platinum-copper-alumina catalyst and a platinum-palladium-alumina catalyst was reduced at 500° C. for 2 hours, and then dehydrogenation of n-butane was performed under the conditions of hydrogen:n-butane:nitrogen=1:1:1, a space velocity (GHSV) of 18000 ml·gcat⁻¹·h⁻¹, a reaction temperature of 550° C. It was reported that a platinum-copper-alumina catalyst using copper as an enhancer resulted in n-butane conversion rate of 17.1% and C4 olefin selectivity of 95.4%, after 5 hours of reaction, and a platinum-palladium-alumina catalyst resulted in n-butane conversion rate of 7.6% and C4 olefin selectivity of 86.7%, after 5 hours of reaction. Further, it is known that when an alkali metal is added to a platinum-tin-alumina catalyst, higher C4 olefin selectivity and yield can be obtained, and as an example thereof, a document which uses sodium as an enhancer in a platinum-tin-alumina catalyst has been reported (Non-patent document 16). In the above document, a platinum-tin-alumina catalyst with added sodium, was prepared by adding sodium to a conventional alumina and supporting platinum and tin thereto, and thus prepared catalyst 0.2 g was reduced at 530° C. for 3 hours by using hydrogen. Next, the platinum-tin-alumina catalyst containing 0.3 wt % sodium was subjected to a dehydrogenation reaction under the conditions of a total flow rate of 18 ml·min⁻¹ and a feed ratio of hydrogen:n-butane=1.25:1, resulting in, after 10 minutes of reaction, n-butane conversion rate of 34% and C4 olefin selectivity of 96%, and after 2 hours of reaction, n-butane conversion rate of 19% and C4 olefin selectivity of 97%.

When a platinum-tin-alumina catalyst, in which platinum and tin has been supported by alumina, is used in a direct dehydrogenation reaction of n-butane, it is possible to obtain C4 olefins with high selectivity and yield; however inactivation occurs owing to coking deposition in the course of the catalyst reaction and thus the high catalyst activity is not maintained for a long term. In this regard, catalysts maintaining its performance for a long period are needed to be developed.

### DOCUMENTS IN PRIOR ARTS

### Patent Document

(Patent document 1) U.S. Pat. No. 6,433,241 B2 (A. Wu, C. A. Drake) Aug. 13, 2002.
(Patent document 2) U.S. Pat. No. 6,187,984 B1 (A. Wu, C. A. Drake) Feb. 13, 2001.
(Patent document 3) U.S. Pat. No. 5,344,805 (G. P. Khare, R. A. Porter) May 3, 1993.
(Patent document 4) U.S. Pat. No. 4,827,072 (T. Imai, H. Abrevaya, J. C. Bricker, D. Jan) Jul. 20, 1988.
(Patent document 5) U.S. Pat. No. 3,960,975 (H. E. Manning) Jun. 3, 1974.
(Patent document 6) U.S. Pat. No. 3,960,776 (M. C. Ellis, H. E. Manning) Jul. 1, 1974.

### [Non-Patent Document]

(Non-patent document 1) S. Wang, K. Murata, T. Hayakawa, S. Hamakawa, K. Suzuki, Catal. Lett., vol. 66, p. 13 (2000).
(Non-patent document 2) A. D. Ballarini, C. G. Ricci, S. R. de Miguel, O. A. Scelza, Catal. Today, vol. 133, p. 28 (2008).
(Non-patent document 3) G. Raju, B. M. Reddy, B. Abhishek, Y.-H. Mo, S.-E. Park, Appl. Catal. A, vol. 423, p. 168 (2012).
(Non-patent document 4) M. Setnicka, R. Bulanek, L. Capek, P. Cicmanec, J. Mol. Catal. A, vol. 344, p. 1 (2010).
(Non-patent document 5) N. Kijima, M. Toba, Y. Yoshimura, Catal. Lett., vol. 127, p. 63 (2009).
(Non-patent document 6) W. Liu, S. Y, Lai, H. Dai, S. Wang, H. Sun, C. T. Au, Catal. Lett., vol. 113, p. 147 (2007).
(Non-patent document 7) M. M. Bhasin, J. H. McCain, B. V. Vora, T. Imai, P. R. Pujado, Appl. Catal. A, vol. 221, p. 397 (2001).
(Non-patent document 8) J. M. McNamara, S. D. Jackson, D. Lennon, Catal. Today, vol. 81, p. 583 (2003).
(Non-patent document 9) S. A. Bocanegra, A. A. Castro, O. A. Scelza, S. R. de Miguel, Appl. Catal. A, vol. 333, p. 49 (2007).
(Non-patent document 10) S. A. Bocanegra, S. R. de Miguel, I. Borbath, J. L. Margitfalvi, S. A. Scelza, J. Mol. Catal. A, vol. 301, p. 52 (2009).
(Non-patent document 11) M. E. Harlin, A. O. I. Krause, B. Heinrich, C. Pham-Huu, M. J. Ledoux, Appl. Catal. A, vol. 185, p. 311 (2012).
(Non-patent document 12) S. D. Jackson, S. Rugmini, J. Catal., vol. 251, p. 59 (2007).
(Non-patent document 13) Y. Xu, J. Lu, M. Zhong, J. Wang, J. Nat. Gas Chem., vol. 18, p. 88 (2009).
(Non-patent document 14) S. Bocanegra, A. Ballarini, P. Zgolicz, O, Scelza, S. de Miguel, Catal. Today, vol. 143, p. 334 (2009).
(Non-patent document 15) S. Veldurthi, C.-H. Shin, O.-S. Joo, K.-D. Jung, Catal. Today, vol. 185, p. 88 (2012).
(Non-patent document 16) S. R. de Miguel, S. A. Bocanegra, I. M. Julieta Vilella, A. Guerrero-Ruiz, O. A. Scelza, Catal. Lett., vol. 119, p. 5 (2007).

### SUMMARY OF THE INVENTION

With a purpose to solve the problem of a decrease in a platinum-tin-alumina catalyst activity over time in prior arts, the present inventors have developed a method of introducing various metals to a platinum-tin-alumina catalyst. In this regard, the present inventors have established a catalyst preparation technique regarding a platinum-tin-metal-alumina catalyst by, before supporting platinum and tin to an alumina carrier, supporting additional other metals to the alumina carrier, and thus developed a catalyst reaction process for C4 olefin production with a high production yield by suppressing catalyst inactivation over reaction time by using the-above prepared catalyst. Further, a method for preparing a platinum-tin-metal-alumina catalyst through a simple process has also been established, thereby ensuring reproducibility in catalyst preparation.

Therefore, the object of the present invention is to provide a simple and reproducible method for preparing a platinum-tin-metal-alumina catalyst by comprising alumina as a carrier, platinum as an active ingredient, tin as a enhancer, and additionally introduced other metals, which can provide high catalyst activity with reduced catalyst inactivation when applied to a direct dehydrogenation reaction of n-butane.

Another object of the present invention is to provide a method for producing C4 olefins by using a platinum-tin-metal-alumina catalyst prepared by the above-described method of the present invention in a direct dehydrogenation of n-butane, which can provide higher activity and suppress catalyst inactivation as compared to the conventional platinum-tin-alumina catalyst.

### DETAILED DESCRIPTION OF THE INVENTION

In order to solve such problems of prior arts, the present invention provides a method for preparing a platinum-tin-metal-alumina catalyst for a direct dehydrogenation reaction of n-butane comprising the following steps:

(a) preparing a solution of a metal precursor by dissolving a metal precursor into a first solvent;

(b) impregnating the metal precursor solution to an alumina carrier;

(c) thermally drying and heat-treating the product obtained from the above step (b) so as to obtain a metal-alumina wherein metal is supported to an alumina carrier;

(d) preparing a tin precursor solution by dissolving a tin precursor and an acid into a second solvent to form a tin precursor solution;

(e) impregnating the above-prepared tin precursor solution from the step (d) to the metal-alumina prepared by the above step (c);

(f) thermally drying and heat-treating the product obtained from the above step (e) to obtain a tin-metal-alumina;

(g) preparing a platinum precursor solution by dissolving a platinum precursor into a third solvent;

(h) impregnating the above-prepared platinum precursor solution from the step (g) to the tin-metal-alumina prepared from the above step (f); and

(i) thermally drying and heat-treating the product obtained from the above step (h) so as to obtain a platinum-tin-metal-alumina catalyst for a direct dehydrogenation reaction of n-butane.

The types of metals used in the above step (a) may be selected from the group consisting of transition metals such a zinc, gallium, indium, lanthanum, cerium and the like, and alkali metals such as lithium, sodium, potassium, rubidium and the like, without being limited to these.

As for the metal precursor used in the above step (a), any conventionally used precursor may be used, and for example at least one selected from metal chloride, nitrate, bromide, oxide, hydroxide or acetate precursors are generally preferred and metal nitrate is particularly preferred.

Although the amount of metal precursors used in the step (a) is not specifically limited, the metal content is preferably 0.2-5 wt %, and more preferably 0.5 wt %, based on the total weight of the final platinum-tin-metal-alumina catalyst, wherein when more than 5 wt % of metal is added thereto, the active sites of platinum may be undesirably blocked during the catalyst preparation, and when the less than 0.2 wt % of metal is added, the amount is not sufficient enough to effect the reactivity increase, disadvantageously.

Each first, second and third solvent used in the step (a), (d) and (g), respectively may be selected from water or alcohols, with a preference to water, however it is not limited thereto.

The alumina used in the step (b) is not specifically limited to certain types, and thus acidic, neutral or basic γ-alumina may be used.

Since the object of the thermal drying process in said step (c) is to remove residual moisture after the impregnation of metal, the drying temperature and time may be defined by general moisture drying conditions, wherein for example, the drying temperature is in the range of 50-200° C., preferably 70-120° C., and drying time is in the range of 3-24 hours, preferably 6-12 hours.

Further, the object of the heat treatment in the step (c) is to form a metal-alumina, and it is carried out in a temperature range of 350-1000° C., preferably 500-800° C. for 1-12 hours and preferably 3-6 hours. In this regard, when the temperature is less than 350° C. or the time is less than 1 hour, the metal-alumina is not sufficiently formed, disadvantageously, when the temperature is more than 1000° C. or the time is more than 12 hours, the metal-alumina phase would be degenerated, undesirably.

Any conventionally used tin precursor may be used in the step (d), among those, preferably used is at least one selected from chloride, nitride, bromide, oxide and acetate precursor, and more preferably used is tin (II) chloride.

Although the amount of the tin precursor used in the step (d) is not specifically limited, the tin content is preferably 0.5-10 wt % and more preferably 1 wt %, based on the total weight of the final platinum-tin-metal-alumina catalyst in order to stably maintain the high catalyst activity for a long time, wherein when more than 10 wt % of tin is added, the number of active sites in platinum is reduced during the catalyst preparation and thus causing a decrease in catalyst activity, disadvantageously, and when less than 0.5 w % is added, the role of tin which prevents the sintering of platinum particles and maintains the platinum particle size small so as to improve the dispersibility and suppress carbon deposition, is not properly effected, disadvantageously.

The acid used in the step (d) is an acid which is present in a liquid (solution) form at room temperature and may be selected at least one from the group consisting of hydrochloric acid, nitric acid, sulfuric acid, hydrofluoric acid and phosphoric acid, without being limited to these examples.

Since the object of the thermal drying in the step (f) is to remove the residual moisture after the impregnation of tin, the drying temperature and time may be defined by general moisture drying conditions, wherein for example, the drying temperature is in the range of 50-200° C., preferably 70-120° C., and drying time is in the range of 3-24 hours, preferably 6-12 hours.

Further, the object of the heat treatment in the step (f) is to form a tin-metal-alumina, and it is carried out in a temperature range of 350-1000° C., preferably 500-800° C. for 1-12 hours and preferably 3-6 hours. In this regard, when the temperature is less than 350° C. or the time is less than 1 hour, the tin-metal-alumina is not sufficiently formed, disadvantageously, when the temperature is more than 1000° C. or the time is more than 12 hours, the tin-metal-alumina phase would be degenerated, undesirably.

Any conventionally used platinum precursor may be used in the step (g), among those, preferably used is at least one selected from chloroplatinic acid, platinum oxide, platinum chloride and platinum bromide precursor, and more preferably used is chloroplatinic acid.

Although the amount of the platinum precursor used in the step (g) is not specifically limited, the platinum content is preferably 0.5-10 wt %, based on the total weight of the final platinum-tin-metal-alumina catalyst, wherein when more than 10 wt % of platinum is added, good dispersion of platinum in catalyst preparation is hardly obtained and it becomes undesirably cost-expensive owing to the much use of expensive platinum, and when less than 0.5 w % is added, the active sites of platinum that is an active metal ingredient in a direct dehydrogenation of n-butane are not sufficiently formed and thus it becomes difficult to prepare C4 olefins with a high selectivity and yield, disadvantageously.

Since the object of the thermal drying in the step (i) is to remove the residual moisture after the impregnation of platinum, the drying temperature and time may be defined by general moisture drying conditions, wherein for example, the drying temperature is in the range of 50-200° C., preferably 70-120° C., and drying time is in the range of 3-24 hours, preferably 6-12 hours.

Further, the heat treatment of the step (i) may be carried out in a temperature range of 400-800° C., for 1-2 hours, and preferably 500-700° C. for 3-6 hours so as to obtain a platinum-tin-metal-alumina catalyst. The heat treatment of the dried solid product is not only to obtain a platinum-tin-metal-alumina catalyst but also to prevent the catalyst from being degenerated during the use of the prepared catalyst in the direct dehydrogenation reaction of n-butane, considering the reaction temperature thereof, wherein when the temperature is less than 400° C. or the time is less than 1 hour, a platinum-tin-metal-alumina catalyst is not properly formed, and when the temperature is more than 800° C. or the time is more than 12 hours, the crystalline phase of the platinum-tin-metal-alumina catalyst would become possibly unsuitable for the use as a catalyst, disadvantageously.

The present invention further provide a method for preparing C4 olefins through a direct dehydrogenation of n-butane by using a platinum-tin-metal-alumina catalyst prepared by the above-described method of the present invention.

The reactants of the direct dehydrogenation of n-butane are n-butane and nitrogen in the form of a mixed gas, wherein the ratio of n-butane:nitrogen by volume is 1:0.2-10, preferably 1:0.5-5, more preferably 1:1, based on n-butane. When the volume ratio of n-butane is out of said range, catalyst inactivation caused by coking of n-butane during the direct dehydrogenation of n-butane may occur rapidly, and the catalyst activity and selectivity, C4 olefin production amount and process safety are lowered, undesirably.

When feeding the reactants in the form of a mixed gas to a reactor, the catalyst amount was the feed amount may be adjusted by using a mass flow controller, wherein the catalyst amount is set to make the feed amount become preferably 10-6000 cc·hr⁻¹·gcat⁻¹, preferably 100-3000 cc·hr⁻¹·gcat⁻¹, more preferably 300-1000 cc·hr⁻¹·gcat⁻¹ of Weight Hourly Space Velocity (WHSV) based on n-butane. When WHSV is less than 10 cc·hr⁻¹·gcat⁻¹, the amount of C4 olefin produced is undesirably too small, and when it is more than 6000 cc·hr⁻¹·gcat⁻¹, coking deposition owing to the side-products from the catalyst reaction rapidly occurs undesirably.

The reaction temperature for the practice of a direct dehydrogenation reaction of n-butane is preferably in the range of 300-800° C., more preferably 500-600° C., and most preferably 550° C. When the reaction temperature is less than 300° C., the dehydrogenation of n-butane reaction is not sufficiently activated, disadvantageously, when it is more than 800° C., a decomposition reaction of n-butane undesirably occurs.

### INDUSTRIAL AVAILABILITY

According to the present invention, it is possible to prepare a platinum-tin-metal-alumina catalyst through a simple method and ensure an excellent reproducibility in catalyst preparation.

Moreover, by using the platinum-tin-metal-alumina catalyst according to the present invention, it is possible to produce C4 olefins of which demand and value are more and more increasing globally, with a high production yield, from n-butane that has a lower value for practical use, and thus to maximize the use of carbon resources.

Further, a single production process for C4 olefin preparation can be ensured by using the platinum-tin-metal-alumina catalyst according to the present invention, which makes possible to fulfill the increasing demand for C4 olefins without further establishment of a naphtha cracking equipment, thereby further obtaining economic benefits.

### BRIEF DESCRIPTION OF THE DRAWINGS

FIG. 1 is a graph showing the differences in the direct dehydrogenation reaction yield between catalysts during the direct dehydrogenation reaction of n-butane on a platinum-tin-alumina catalyst and 5 species of platinum-tin-transition metal-alumina catalysts according to Examples of the present invention for 360 minutes.

FIG. 2 is a graph showing the differences in the direct dehydrogenation reaction yield between catalysts after the direct dehydrogenation reaction of n-butane on a platinum-tin-alumina catalyst and 5 species of platinum-tin-transition metal-alumina catalysts according to Examples of the present invention for 360 minutes.

FIG. 3 is a graph showing the differences in the direct dehydrogenation reaction yield between catalysts during the direct dehydrogenation reaction of n-butane on 4 species of platinum-tin-alkali metal-alumina catalysts according to Examples of the present invention for 360 minutes.

FIG. 4 is a graph showing the differences in the direct dehydrogenation reaction yield between catalysts during the direct dehydrogenation reaction of n-butane on 3 species of platinum-tin-alkali earth metal-alumina catalysts according to Comparative examples for 360 minutes.

### EXAMPLES OF THE INVENTION

Hereinafter, the present invention is further described in detail through specific embodiments. However, these examples are provided only with an illustrative purpose without any intention to limit the present invention.

### Preparation Example 1

### Preparation of Zinc-Alumina (Zn—Al₂O₃) Through an Impregnation of Zinc by Using a Conventional Alumina Carrier

For preparing Zn—Al₂O₃ in which zinc was supported to the content of 0.5 wt % on a conventional alumina carrier (γ-Alumina, surface area=180 m²/g), 0.046 g of zinc nitrate hexahydrate was placed in a beaker and dissolved in distilled water therein. To thus prepared solution, when the precursor was completely dissolved, 2.0 g of conventional alumina was placed thereto, and the resulted mixture was heated at 70° C. with stirring until distilled water was completely evaporated, resulting in a solid product. After that, the solid product was additionally dried in an oven at a temperature of 80° C. for about 12 hours, and thus obtained sample was heat-treated in an electric furnace maintained at a temperature of 600° C. in an air atmosphere for 4 hours so as to form a zinc-alumina product, wherein 0.5% of zinc was supported to alumina. The resulted product was referred as Zn—Al₂O₃.

### Preparation Example 2

### Preparation of Transition Metal-Alumina (M—Al₂O₃) Through an Impregnation of Various Transition Metal (Ga, In, La, Ce) by Using a Conventional Alumina Carrier

According to the above method described in the preparation example 1, various transition metals were used to prepare 4 species of transition metal-alumina. Specifically, as for the various transition metal, gallium, indium, lanthanum, cerium were used, and as for the precursors, gallium(III) nitrate hydrate, indium(III) nitrate hydrate, lanthanum (III) nitrate hexahydrate and cerium(III) nitrate hexahydrate were used, respectively.

After adjusting the metal content to become 0.5 wt %, it was impregnated so as to form a solid material, which was dried at 80° C. for about 12 hours, and heat-treated in an electric furnace maintained at a temperature of 600° C. in an air atmosphere for 4 hours, thereby preparing 4 species of transition metal-alumina catalysts in which each transition metal was supported to the amount of 0.5 wt %. The resulted products were referred as Ga—Al₂O₃, In—Al₂O₃, La—Al₂O₃, Ce—Al₂O₃, respectively.

### Preparation Example 3

### Preparation of a Platinum-Tin-Alumina (Pt—Sn—Al₂O₃) Catalyst and a Platinum-Tin-Metal-Alumina (Pt—Sn-M-Al₂O₃) Catalyst Through a Sequential Impregnation of Various Metals, and Tin and Platinum by Using a Conventional Alumina Carrier

A platinum-tin-metal-alumina (Pt—Sn-M-Al₂O₃) catalyst was prepared by the sequential impregnation of tin and platinum to the metal-alumina prepared by the above preparation examples 1 and 2. For comparison, a platinum-tin-alumina catalyst was prepared by sequential impregnation of tin and platinum to alumina.

The preparation of the platinum-tin-metal-alumina catalyst and the platinum-tin-alumina catalyst through impregnation of each tin and platinum to metal-alumina and alumina, respectively were as follows.

For preparing each of a tin-metal-alumina catalyst and a tin-alumina catalyst, in which tin is supported to the content of 1 wt %, by using a metal-alumina and alumina, tin (II) chloride dihydrate 0.038 g was placed in a beaker and dissolved into a small amount of hydrochloric acid 0.37 ml and distilled water 15 ml. When the precursor solution was completely dissolved, 2.0 g of each metal-alumina and alumina previously prepared according to the above preparation examples 1 and 2 was placed thereto, and the resulted mixture was heated at 70° C. with stirring until distilled water was completely evaporated. After that, the remained solid product was additionally dried in an oven at a temperature of 80° C. for about 12 hours, and thus obtained sample was heat-treated in an electric furnace maintained at a temperature of 600° C. in an air atmosphere for 4 hours so as to form each of a tin-metal-alumina (Sn-M-Al₂O₃) and tin-alumina (Sn—Al₂O₃) in which 1 wt % of tin was supported.

To thus obtained tin-metal-alumina and tin-alumina product 2.0 g, chloroplatinic acid hexahydrate 0.053 g was placed in a beaker and dissolved into 10 ml distilled water so that the platinum content became 1 wt %. When the platinum precursor solution was completely dissolved, 2.0 g of each previously prepared tin-metal-alumina and tin-alumina was placed thereto, and the resulted mixture was heated at 70° C. with stirring until distilled water was completely evaporated. After that, if any, solid product remained was additionally dried in an oven at a temperature of 80° C. for about 12 hours, and thus obtained sample was heat-treated in an electric furnace maintained at a temperature of 550° C. in an air atmosphere for 4 hours so as to form a platinum-tin-metal-alumina catalyst and a platinum-tin-alumina catalyst, wherein the finally prepared catalysts were referred as Pt—Sn—Zn—Al₂O₃, Pt—Sn—Ga—Al₂O₃, Pt—Sn—In—Al₂O₃, Pt—Sn—La—Al₂O₃, Pt—Sn—Ce—Al₂O₃, respectively, according to the species of metal used therein, and the catalyst having no added metal was referred as Pt—Sn—Al₂O₃.

### Preparation Example 4

### Preparation of a Platinum-Tin-Alkali Metal-Alumina (Pt—Sn-M-Al₂O₃) Catalyst Through a Sequential Impregnation of Various Alkali Metals, and Tin and Platinum by Using a Conventional Alumina Carrier

According to the above method described in the preparation examples 1 and 2, various alkali metals, and tin and platinum were sequentially impregnated to prepare 4 species of platinum-tin-alkali metal-alumina. Specifically, each alkali metal was impregnated to alumina to form an alkali metal-alumina product, wherein as for the alkali metal, lithium, sodium, potassium and rubidium were used, and as for the precursors, lithium nitrate, sodium nitrate, potassium nitrate and rubidium nitrate were used, respectively. To the prepared alkali metal-alumina, tin and platinum were sequentially impregnated according to the preparation example 3 so as to form a platinum-tin-alkali metal-alumina catalyst, and each catalyst was referred as Pt—Sn—Li—Al₂O₃, Pt—Sn—Na—Al₂O₃, Pt—Sn—K—Al₂O₃, Pt—Sn—Rb—Al₂O₃, according to the species of metal used therein.

### Preparation Example 5 (Comparative Preparation Example)

### Preparation of a Platinum-Tin-Alkali Earth Metal-Alumina (Pt—Sn-M-Al₂O₃) Catalyst Through a Sequential Impregnation of Various Alkali Earth Metals, and Tin and Platinum by Using a Conventional Alumina Carrier

According to the above method described in the preparation examples 1 and 2, various alkali earth metals, and tin and platinum were sequentially impregnated to prepare 3 species of platinum-tin-alkali earth metal-alumina. Specifically, each alkali earth metal was impregnated to alumina to form an alkali earth metal-alumina product, wherein as for the alkali earth metal, magnesium, calcium and barium were used, and as for the precursor, magnesium nitrate hexahydrate, calcium nitrate tetrahydrate and barium nitrate were used, respectively. To the prepared alkali earth metal-alumina, and tin and platinum were sequentially impregnated according to the preparation example 3 so as to form a platinum-tin-alkali earth metal-alumina catalyst, and each catalyst was referred as Pt—Sn—Mg—Al₂O₃, Pt—Sn—Ca—Al₂O₃, Pt—Sn—Ba—Al₂O₃, according to the species of metal used therein.

### Example 1

### Direct Dehydrogenation Reaction in a Continuous Flow Catalyst Reactor

A direct dehydrogenation reaction was conducted by using the platinum-tin-zinc-alumina catalyst prepared from the above preparation example 3.

The reactant used in the direct dehydrogenation reaction of n-butane in this example was a C4 mixture including 99.65 wt % of n-butane, and specific composition thereof was presented in the following Table 1.

TABLE 1
Composition of the C4 mixture used as a reactant

| composition | molecular formula | wt % |
|---|---|---|
| n-butane | C₄H₁₀ | 99.65 |
| i-butane | C₄H₁₀ | 0.27 |
| 1-butane | C₄H₈ | 0.03 |
| cis-2-butane | C₄H₈ | 0.05 |

| | |
|---|---|
| total | 100.00 |

For a catalyst reaction, a linear type quartz reactor was equipped in an electric furnace and packed with said catalyst, and then a reduction process was carried out for catalyst activation before beginning the reaction. In the reduction process, the temperature of the fixed bed reactor was elevated from room temperature to 570° C. and maintained at 570° C. for 3 hours; a gas mixture of hydrogen and nitrogen at a mixing ratio of 1:1 was fed for the reduction process; and the catalyst amount for the reaction was set to make the feeding rate become 600 cc·hr⁻¹·gcat⁻¹ based on hydrogen.

Next, the reactor temperature was lowered to 550° C., a C 4 mixture comprising n-butane and nitrogen was passed through the catalyst bed to carry out a direct dehydrogenation reaction of n-butane. At this time, as for a gas for the reaction, the ratio of n-butane:nitrogen at a mixing ratio of 1:1 was fed, and the feeding rate was set to be 600 cc·hr⁻¹·gcat⁻¹ based on the adjusted catalyst amount and n-butane.

After finishing the reaction, there were: a major product, i.e. C4 olefins such as 1-butene, 2-butene, i-butene and 1,3-butadiene; side products, other than said major product, including those from cracking such as methane, ethane, ethylene, propane, propylene and those from isomerization such as i-butane and the like; and unreacted n-butane, and for separating and analyzing them, gas chromatography was used.

In a direct dehydrogenation reaction of n-butane on a platinum-tin-zinc-alumina catalyst, the n-butane conversion rate, C4 olefin selectivity and C4 olefin yield were calculated using the following formulas I to 3.

$$\begin{matrix}conversion rate ( \% ) = \frac{mole number of reacted n \text{-} butane}{mole number of fed n \text{-} butane} \times 100 & [ Formula 1 ] \\ selectivity ( \% ) = \frac{mole number of produced C 4 olefin}{mole number of reacted n \text{-} butane} & [ Formula 2 ] \\ yield ( \% ) = \frac{mole number of produced C 4 olefin}{mole number of fed n \text{-} butane} \times 100 & [ Formula 3 ]\end{matrix}$$

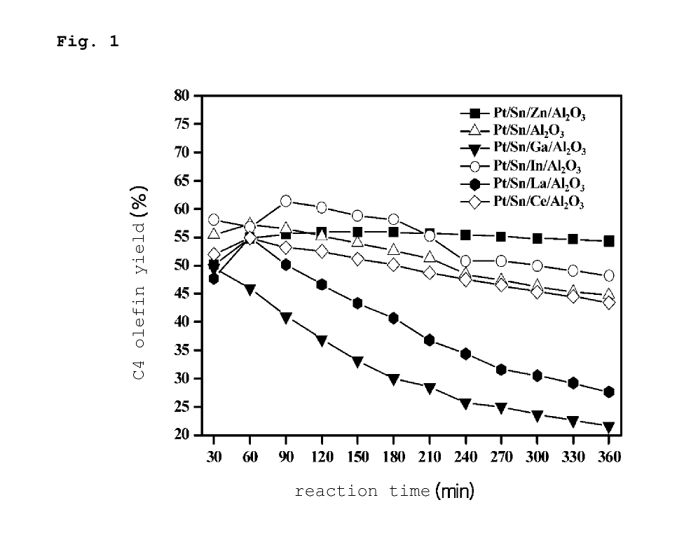

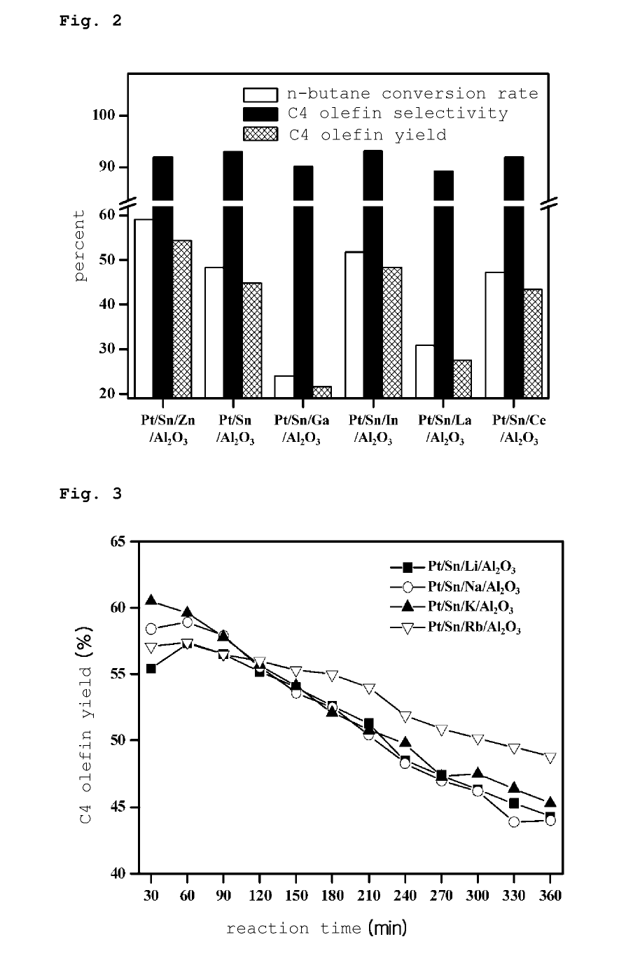

Direct dehydrogenation reaction was performed on the platinum-tin-zinc-alumina catalyst obtained from the preparation examples 1 and 2 for 360 minutes, and the change in a reactivity throughout the process was shown in Table 2 and the change in C4 olefin yield was shown in FIG. 1. Further, the test results regarding reactivity after 360 minutes of the reaction were shown in Table 3 and FIG. 2.

TABLE 2
Change in the reactivity in a direct
dehydrogenation reaction of a platinum-tin-zinc-alumina
(Pt—Sn—Zn—Al₂O₃) catalyst for 360 minutes

| | n-butane | C4 | |
|---|---|---|---|
| | conversion | olefin selectivity | |
| reaction time (min) | rate (%) | (%) | C4 olefin yield (%) |
| 30 | 70.2 | 72.5 | 50.2 |
| 60 | 65.8 | 84.5 | 54.9 |
| 90 | 59.2 | 87.0 | 55.5 |
| 120 | 66.2 | 87.0 | 56.0 |
| 150 | 64.3 | 87.1 | 56.0 |
| 180 | 63.2 | 88.4 | 55.9 |
| 210 | 62.3 | 89.2 | 55.7 |
| 240 | 61.5 | 90.1 | 55.4 |
| 270 | 60.8 | 90.7 | 55.1 |
| 300 | 60.2 | 91.1 | 54.8 |
| 330 | 59.6 | 91.5 | 54.6 |
| 360 | 59.1 | 91.8 | 54.3 |

TABLE 3
Reactivity in a direct dehydrogenation reaction of
a platinum-tin-zinc-alumina (Pt—Sn—Zn—Al₂O₃) catalyst after
360 minutes

| | | percent (%) | |
|---|---|---|---|
| n-butane conversion rate | | 59.1 | |
| selectivity | 1-butene | 24.2 | 91.9 |
| | 2-butene | 54.9 | |
| | i-butene | 7.1 | |
| | 1,3-butadiene | 5.7 | |

| | |
|---|---|
| i-butane | 1.5 |
| methane | 1.1 |
| ethane | 1.9 |
| ethylene | 0.1 |
| propane | 1.1 |
| propylene | 0.7 |
| C4 olefin yield | 54.3 |

From Tables 2 and 3, and FIGS. 1 and 2, the direct dehydrogenation of n-butane using a Pt—Sn—Zn—Al₂O₃ catalyst showed a tendency of a gradual decrease in activation as time elapses (resulting in a conversion rate and yield decrease), contrarily an increase in selectivity. As it has been reported by various documents, it seemed that inactivation occurred owing to coking deposition. The selectivity for C4 olefins such as 1-butene, 2-butene, i-butene and 1,3-butadiene was as high as about 90% or more, and major side products were shown to be cracking products such as methane, ethane, propane and propylene.

### Example 2

### The Reactivity of a Platinum-Tin-Alumina Catalyst and Platinum-Tin-Transition Metal-Alumina Catalysts (Pt—Sn—Al₂O₃, Pt—Sn—Ga—Al₂O₃, Pt—Sn—In—Al₂O₃, Pt—Sn—La—Al₂O₃, Pt—Sn—Ce—Al₂O₃) Prepared by the Above Preparation Example 3 in a Direct Dehydrogenation Reaction

For comparing with the results from the reactivity of direct dehydrogenation reaction of n-butane using the platinum-tin-zinc-alumina (Pt—Sn—Zn—Al₂O₃) catalyst prepared by using conventional alumina carrier (γ-Alumina) according to Example 1, a direct dehydrogenation reaction of n-butane using the platinum-tin-transition metal-alumina catalyst (Pt—Sn—Al₂O₃, Pt—Sn—Ga—Al₂O₃, Pt—Sn—In—Al₂O₃, Pt—Sn—La—Al₂O₃, Pt—Sn—Ce—Al₂O₃) prepared by impregnating various transition metals to a conventional alumina (γ-Alumina) according to Preparation example 3 was carried out after the reduction process according to the sequence of Example 1.

The reactivity test results from the present example 2 were shown in Tables 4-9 and FIGS. 1 and 2; change in reactivity throughout the reaction over 360 minutes regarding each catalyst was shown in Table 4 (Pt—Sn—Al₂O₃ catalyst), Table 5 (Pt—Sn—Ga—Al₂O₃ catalyst), Table 6 (Pt—Sn—In—Al₂O₃ catalyst), Table 7 (Pt—Sn—La—Al₂O₃ catalyst), Table 8 (Pt—Sn—Ce—Al₂O₃ catalyst); change in C4 olefin production yield from said 5 species of catalysts, respectively over the reaction for 360 minutes was shown FIG. 1; and the reactivity results after the reaction for 360 minutes were shown in Table 9 and FIG. 2.

TABLE 4
Change in a reactivity in a direct dehydrogenation
reaction of a platinum-tin-alumina (Pt—Sn—Zn—Al₂O₃) catalyst
for 360 minutes

| | n-butane | C4 | |
|---|---|---|---|
| | conversion | olefin selectivity | |
| reaction time (min) | rate (%) | (%) | C4 olefin yield (%) |
| 30 | 73.8 | 75.0 | 55.4 |
| 60 | 68.9 | 83.1 | 57.3 |
| 90 | 65.5 | 86.3 | 56.5 |
| 120 | 62.6 | 88.2 | 55.2 |
| 150 | 60.4 | 89.5 | 54.0 |
| 180 | 58.1 | 90.4 | 52.6 |
| 210 | 56.3 | 91.1 | 51.3 |
| 240 | 52.8 | 91.8 | 48.5 |
| 270 | 51.5 | 92.2 | 47.4 |
| 300 | 50.0 | 92.6 | 46.3 |
| 330 | 48.9 | 92.7 | 45.3 |
| 360 | 48.2 | 92.9 | 44.8 |

TABLE 5
Change in a reactivity in a direct dehydrogenation
reaction of a platinum-tin-gallium-alumina (Pt—Sn—Ga—Al₂O₃)
catalyst for 360 minutes

| | n-butane | C4 | |
|---|---|---|---|
| | conversion | olefin selectivity | |
| reaction time (min) | rate (%) | (%) | C4 olefin yield (%) |
| 30 | 57.2 | 82.4 | 49.5 |
| 60 | 51.7 | 88.9 | 46.0 |
| 90 | 45.3 | 90.6 | 41.0 |
| 120 | 40.5 | 91.2 | 37.0 |
| 150 | 36.4 | 91.2 | 33.2 |
| 180 | 32.8 | 91.3 | 30.0 |
| 210 | 31.1 | 91.3 | 28.5 |
| 240 | 29.2 | 91.1 | 25.7 |
| 270 | 27.5 | 90.9 | 25.0 |
| 300 | 26.1 | 90.5 | 23.6 |
| 330 | 25.0 | 90.5 | 22.6 |
| 360 | 24.0 | 90.1 | 21.6 |

TABLE 6
Change in a reactivity in a direct dehydrogenation
reaction of a platinum-tin-indium-alumina (Pt—Sn—In—Al₂O₃)
catalyst for 360 minutes

| | n-butane | | |
|---|---|---|---|
| reaction time | conversion rate | C4 olefin | C4 olefin |
| (min) | (%) | selectivity(%) | yield(%) |
| 30 | 78.0 | 74.4 | 58.1 |
| 60 | 69.7 | 81.6 | 56.8 |
| 90 | 71.4 | 86.0 | 61.4 |
| 120 | 68.9 | 87.5 | 60.3 |
| 150 | 66.7 | 88.2 | 58.8 |
| 180 | 64.6 | 90.1 | 58.2 |
| 210 | 60.7 | 91.0 | 55.2 |
| 240 | 55.1 | 92.1 | 50.8 |
| 270 | 55.1 | 92.2 | 50.8 |
| 300 | 54.0 | 92.5 | 50.0 |
| 330 | 53.0 | 92.8 | 49.1 |
| 360 | 51.8 | 93.0 | 48.2 |

TABLE 7
Change in a reactivity in a direct dehydrogenation
reaction of a platinum-tin-lanthanum-alumina (Pt—Sn—La—Al₂O₃)
catalyst for 360 minutes

| | n-butane | C4 | |
|---|---|---|---|
| | conversion | olefin selectivity | |
| reaction time (min) | rate (%) | (%) | C4 olefin yield (%) |
| 30 | 61.0 | 78.2 | 47.7 |
| 60 | 65.8 | 84.5 | 54.9 |
| 90 | 59.2 | 87.0 | 50.2 |
| 120 | 52.4 | 89.1 | 46.6 |
| 150 | 48.1 | 90.1 | 43.3 |
| 180 | 45.0 | 90.3 | 40.7 |
| 210 | 40.7 | 90.3 | 36.8 |
| 240 | 38.1 | 90.3 | 34.4 |
| 270 | 35.2 | 89.8 | 31.6 |
| 300 | 34.0 | 89.8 | 30.5 |
| 330 | 32.6 | 89.6 | 29.2 |
| 360 | 30.9 | 89.3 | 27.6 |

TABLE 8
Change in a reactivity in a direct dehydrogenation
reaction of a platinum-tin-cerium-alumina (Pt—Sn—Ce—Al₂O₃)
catalyst for 360 minutes

| | n-butane | C4 | |
|---|---|---|---|
| | conversion | olefin selectivity | |
| reaction time (min) | rate (%) | (%) | C4 olefin yield (%) |
| 30 | 68.0 | 76.5 | 52.0 |
| 60 | 65.8 | 84.5 | 54.9 |
| 90 | 59.2 | 87.0 | 53.2 |
| 120 | 59.2 | 88.7 | 52.5 |
| 150 | 57.0 | 89.8 | 51.2 |
| 180 | 55.4 | 90.4 | 50.1 |
| 210 | 53.5 | 91.1 | 48.7 |
| 240 | 52.0 | 91.3 | 47.5 |
| 270 | 50.7 | 91.5 | 46.5 |
| 300 | 49.4 | 91.8 | 45.3 |
| 330 | 48.4 | 91.9 | 44.5 |
| 360 | 47.3 | 91.9 | 43.4 |

TABLE 9
Reactivity in a direct dehydrogenation reaction of a platinum-tin-alumina and platinum-tin-
transition metal-alumina (Pt—Sn—Zn—Al₂O₃, Pt—Sn—Ga—Al₂O₃, Pt—Sn—In—Al₂O₃,
Pt—Sn—La—Al₂O₃, Pt—Sn—Ce—Al₂O₃) catalysts after 360 minutes

| | | catalyst | | | | | | | | | |
|---|---|---|---|---|---|---|---|---|---|---|---|
| | | Pt/Sn/ | | Pt/Sn/Ga/ | | Pt/Sn/In/ | | Pt/Sn/La/ | | Pt/Sn/Ce/ | |
| | | Al₂O₃ | | Al₂O₃ | | Al₂O₃ | | Al₂O₃ | | Al₂O₃ | |
| n-butane conversion rate (%) | | 48.2 | | 24.0 | | 51.8 | | 30.9 | | 47.3 | |
| selectivity | 1-butene | 22.3 | 92.9 | 20.6 | 90.1 | 22.4 | 93.0 | 20.6 | 89.3 | 23.1 | 91.9 |
| (%) | 2-butene | 48.9 | | 45.5 | | 49.3 | | 47 | | 52.5 | |
| | i-butene | 14.6 | | 12.9 | | 14.2 | | 14.2 | | 10.3 | |
| | 1,3-butadiene | 7.1 | | 11.1 | | 7.1 | | 7.5 | | 6.0 | |

| | | | | | |
|---|---|---|---|---|---|
| i-butane | 1.6 | 0.4 | 1.6 | 0.4 | 0.9 |
| methane | 0.9 | 1.6 | 0.9 | 1.7 | 1.2 |
| ethane | 1.6 | 1.6 | 1.4 | 2.3 | 2.0 |
| ethylene | 0.4 | 1.5 | 0.4 | 1.0 | 0.3 |
| propane | 0.8 | 0.5 | 0.8 | 0.9 | 1.4 |
| propylene | 1.4 | 3.9 | 1.4 | 4.0 | 1.9 |

| | | | | | |
|---|---|---|---|---|---|
| C4 olefin yield (%) | 44.8 | 21.6 | 48.2 | 27.6 | 43.4 |

From Tables 4-9 and FIGS. 1 and 2, in the catalyst activity test performed by using each catalyst, all of the catalysts showed a tendency of a gradual decrease in activation as time elapses (resulting in a conversion rate and yield decrease), contrarily an increase in selectivity. It can be found out that the Pt—Sn—Al₂O₃ catalyst prepared by sequentially impregnating zinc, and tin and platinum to a conventional alumina (γ-Alumina) showed higher activity than other 5 species of catalysts i.e, Pt—Sn—Al₂O₃, Pt—Sn—Ga—Al₂O₃, Pt—Sn—In—Al₂O₃, Pt—Sn—La—Al₂O₃, Pt—Sn—Ce—Al₂O₃, and also showed a lower inactivation in the elapse of time. Therefore, the Pt—Sn—Zn—Al₂O₃ catalyst prepared by sequentially impregnating zinc, and tin and platinum to a conventional alumina carrier (γ-Alumina) according to the present invention was considered to be most suitable for a catalyst for a direct dehydrogenation of n-butane.

### Example 3

### The Reactivity of a Platinum-Tin-Alkali Metal-Alumina Catalysts (Pt—Sn—Li—Al₂O₃, Pt—Sn—Na—Al₂O₃, Pt—Sn—K—Al₂O₃, Pt—Sn—Rb—Al₂O₃) Prepared by the Above Preparation Example 4 in a Direct Dehydrogenation Reaction

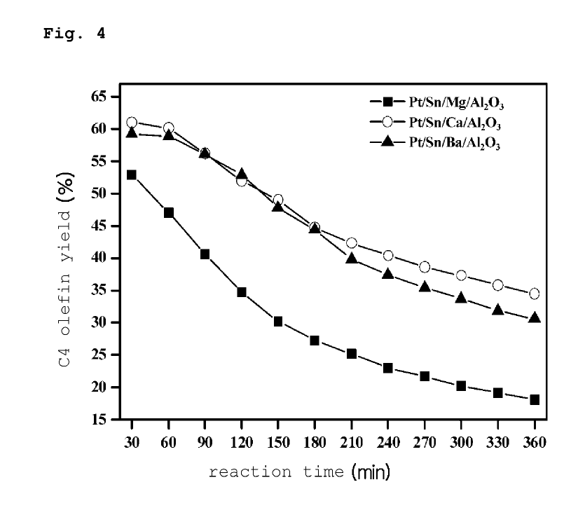

A direct dehydrogenation reaction of n-butane using each Pt—Sn—Li—Al₂O₃, Pt—Sn—Na—Al₂O₃, Pt—Sn—K—Al₂O₃ and Pt—Sn—Rb—Al₂O₃ catalyst prepared by sequentially impregnating alkali metal, and tin and platinum to a conventional alumina (γ-Alumina) according to Preparation example 4 was carried out according to the sequence of Example 1. The reaction results of the present example 3 were shown as change in the yield from a direct dehydrogenation reaction of n-butane, for each catalyst over the elapse of time, in Table 10 and FIG. 3.

TABLE 10
Change in the reactivity in a direct dehydrogenation reaction of a platinum-tin-alkali
metal-alumina catalysts (Pt—Sn—Li—Al₂O₃, Pt—Sn—Na—Al₂O₃, Pt—Sn—K—Al₂O₃,
Pt—Sn—Rb—Al₂O₃, Pt—Sn—Ce—Al₂O₃) catalysts for 360 minutes

| | C4 olefin product yield (%) | | | | | | | | | | | |
|---|---|---|---|---|---|---|---|---|---|---|---|---|
| | time (minutes) | | | | | | | | | | | |
| catalyst | 30 | 60 | 90 | 120 | 150 | 180 | 210 | 240 | 270 | 300 | 330 | 360 |
| Pt/Sn/Li/Al₂O₃ | 55.4 | 57.3 | 56.5 | 55.2 | 54.0 | 52.6 | 51.3 | 48.5 | 47.4 | 46.3 | 45.3 | 44.3 |
| Pt/Sn/Na/Al₂O₃ | 58.4 | 58.9 | 57.9 | 55.5 | 53.6 | 52.5 | 50.4 | 48.3 | 47.0 | 46.2 | 43.9 | 44.0 |
| Pt/Sn/K/Al₂O₃ | 60.5 | 59.6 | 57.8 | 55.7 | 54.1 | 52.1 | 50.8 | 49.8 | 47.3 | 47.5 | 46.4 | 45.3 |
| Pt/Sn/Rb/Al₂O₃ | 57.1 | 57.4 | 56.5 | 56.0 | 55.3 | 55.0 | 54 | 51.9 | 50.9 | 50.2 | 49.5 | 48.8 |

From Table 10 and FIG. 3, in the direct dehydrogenation of n-butane using a platinum-tin-alkali metal-alumina catalyst prepared by sequentially impregnating an alkali metal (lithium, sodium, potassium, rubidium, respectively), and tin and platinum, the Pt—Sn—Rb—Al₂O₃ catalyst showed a high yield and lower inactivation.

### Example 4 (Comparative Example)

### The Reactivity of a Platinum-Tin-Alkali Earth Metal-Alumina Catalyst (Pt—Sn—Mg—Al₂O₃, Pt—Sn—Ca—Al₂O₃ and Pt—Sn—Ba—Al₂O₃) Prepared by the Above Preparation Example 5 (Comparative Example) in a Direct Dehydrogenation Reaction

A direct dehydrogenation reaction of n-butane using each Pt—Sn—Mg—Al₂O₃, Pt—Sn—Ca—Al₂O₃ and Pt—Sn—Ba—Al₂O₃ catalyst prepared by sequentially impregnating alkali earth metal, and tin and platinum to a conventional alumina (γ-Alumina) according to Preparation example 5 was carried out according to the sequence of Example 1. The reaction results of the present example 4 were shown as change in the yield from a direct dehydrogenation reaction of n-butane, for each catalyst over the elapse of time, in Table 11 and FIG. 4.

TABLE 11
Change in the yield of C4 olefin production in a direct dehydrogenation reaction of a
platinum-tin-alkali earth metal-alumina (Pt—Sn—Mg—Al₂O₃, Pt—Sn—Ca—Al₂O₃,
Pt—Sn—Ba—Al₂O₃) catalysts for 360 minutes

| | Yield of C4 olefin products (%) | | | | | | | | | | | |
|---|---|---|---|---|---|---|---|---|---|---|---|---|
| | time (min) | | | | | | | | | | | |
| catalyst | 30 | 60 | 90 | 120 | 150 | 180 | 210 | 240 | 270 | 300 | 330 | 360 |
| Pt/Sn/Mg/Al₂O₃ | 52.9 | 47.1 | 40.6 | 34.7 | 30.2 | 27.3 | 25.2 | 23.0 | 21.7 | 20.3 | 19.2 | 18.1 |
| Pt/Sn/Ca/Al₂O₃ | 61.0 | 60.2 | 56.2 | 52.0 | 49.0 | 44.7 | 42.3 | 40.4 | 38.6 | 37.3 | 35.8 | 34.5 |
| Pt/Sn/Ba/Al₂O₃ | 59.2 | 58.9 | 56.1 | 52.9 | 47.8 | 44.4 | 39.8 | 37.4 | 35.4 | 33.7 | 31.9 | 30.6 |

From Table 11 and FIG. 4, it was confirmed that each C4 olefin yield was low, on the whole, in the direct dehydrogenation of n-butane using a platinum-tin-alkali earth metal-alumina catalyst prepared by sequentially applying each magnesium, calcium and barium as an alkali earth metal, tin and platinum. Initial yields thereof were similar to those of the catalysts from preparation example 3 and 4, however it significantly dropped after 360 minutes indicating the significant inactivation.

1. A method for preparing a platinum-tin-metal-alumina catalyst for a direct dehydrogenation reaction of n-butane comprising the following steps:
(a) preparing a solution of a metal precursor by dissolving a metal precursor into a first solvent;
(b) impregnating the metal precursor solution to an alumina carrier;
(c) thermally drying and heat-treating the product obtained from the above step (b) so as to obtain a metal-alumina wherein metal is supported to an alumina carrier;
(d) preparing a tin precursor solution by dissolving a tin precursor and an acid into a second solvent to form a tin precursor solution;
(e) impregnating the above-prepared tin precursor solution from the step (d) to the metal-alumina prepared by the above step (c);
(f) thermally drying and heat-treating the product obtained from the above step (e) to obtain a tin-metal-alumina;
(g) preparing a platinum precursor solution by dissolving a platinum precursor into a third solvent;
(h) impregnating the above-prepared platinum precursor solution from the step (g) to the tin-metal-alumina prepared from the above step (f); and
(i) thermally drying and heat-treating the product obtained from the above step (h) so as to obtain a platinum-tin-metal-alumina catalyst for a direct dehydrogenation reaction of n-butane.

2. The method according to claim 1, wherein the metal used in step (a) is at least one selected from transition metals and alkali metals.

3. The method according to claim 1, wherein the metal used in step (a) is at least one selected from zinc, gallium, indium, lanthanum, cerium, lithium, sodium, potassium and rubidium.

4. The method according to claim 1, wherein the metal precursor used in the above step (a) is at least one selected from metal chloride, nitrate, bromide, oxide, hydroxide or acetate precursor.

5. The method according to claim 1, wherein the metal content of the step (a) is 0.2-5 wt %, based on the total weight of the finally obtained platinum-tin-metal-alumina catalyst.

6. The method according to claim 1, wherein each first, second and third solvent used in the above step (a), (d) and (g), respectively is water or an alcohol.

7. The method according to claim 1, wherein, in the step (c), the thermal drying is carried out at a temperature range of 50-200° C., and the heat treatment is carried out at a temperature range of 350-1000° C.

8. The method according to claim 1, wherein, in the step (f) and (i), the thermal drying is carried out at a temperature range of 50-200° C., and the heat treatment is carried out at a temperature range of 400-800° C.

9. A method for producing C4 olefins by conducting a direct dehydrogenation reaction of n-butane by using a mixed gas comprising n-butane and nitrogen as reactants on a platinum-tin-metal-alumina catalyst prepared by the method according to claim 1.

10. The method for producing C4 olefins according to claim 9, wherein the direct dehydrogenation reaction of n-butane is carried out at a temperature range of 300-800° C.

11. The method for producing C4 olefins according to claim 9, wherein n-butane:nitrogen ratio by volume in the mixed gas is 1:0.2-10.

12. The method for producing C4 olefins according to claim 9, wherein the feeding amount of the mixed gas is a space velocity of 10-6000 cc·hr⁻¹·gcat⁻¹ based on n-butane.

13. A method for producing C4 olefins by conducting a direct dehydrogenation reaction of n-butane by using a mixed gas comprising n-butane and nitrogen as reactants on a platinum-tin-metal-alumina catalyst prepared by the method according claim 2.

14. A method for producing C4 olefins by conducting a direct dehydrogenation reaction of n-butane by using a mixed gas comprising n-butane and nitrogen as reactants on a platinum-tin-metal-alumina catalyst prepared by the method according claim 3.

15. A method for producing C4 olefins by conducting a direct dehydrogenation reaction of n-butane by using a mixed gas comprising n-butane and nitrogen as reactants on a platinum-tin-metal-alumina catalyst prepared by the method according claim 4.

16. A method for producing C4 olefins by conducting a direct dehydrogenation reaction of n-butane by using a mixed gas comprising n-butane and nitrogen as reactants on a platinum-tin-metal-alumina catalyst prepared by the method according claim 5.

17. A method for producing C4 olefins by conducting a direct dehydrogenation reaction of n-butane by using a mixed gas comprising n-butane and nitrogen as reactants on a platinum-tin-metal-alumina catalyst prepared by the method according claim 6.

18. A method for producing C4 olefins by conducting a direct dehydrogenation reaction of n-butane by using a mixed gas comprising n-butane and nitrogen as reactants on a platinum-tin-metal-alumina catalyst prepared by the method according claim 7.

19. A method for producing C4 olefins by conducting a direct dehydrogenation reaction of n-butane by using a mixed gas.
